(19) United States
(12) Patent Application Publication
(10) Pub. No.: US 2025/0052637 A1
(43) Pub. Date: Feb. 13, 2025

(54) METHOD FOR LEAK SOURCE LOCATION INVESTIGATION

(71) Applicant: Molex, LLC, Lisle, IL (US)

(72) Inventors: Wenfeng Peng, North Aurora, IL (US); Brian Michael Zappa, Wood Dale, IL (US); Ling-Ying Lin, Bloomingdale, IL (US)

(21) Appl. No.: 18/718,438

(22) PCT Filed: Jan. 20, 2023
(86) PCT No.: PCT/US2023/011192

§ 371 Date: Jun. 11, 2024

Related U.S. Application Data
(60) Provisional application No. 63/301,494, filed on Jan. 21, 2022.

(51) Int. Cl. G01M 3/22; H04Q 9/00

(52) U.S. Cl. CPC G01M 3/22; H04Q 9/00; H04Q 2209/40; H04Q 2209/823

(57) ABSTRACT

Methods and systems are disclosed for inspection of a potential source location (PSL) at an industrial facility that has physical components, such as pipes and valves, that transport one or more gaseous materials. A computing device assist in refocusing a technician equipped with a network of mobile sensors and a handheld device/wand to detect a gaseous emission at the industrial facility. The mobile sensors capture measurements on a recurring basis to assist the technician responsible for operating the handheld device/wand to identify exactly which physical component located within a PSL at the industrial facility are causing fugitive emissions. The disclosed system and method update an initial PSL by considering the recurring measurements and/or other inputs (e.g., weather data) to reduce the area of the initial PSL, thus better instructing the mechanic to identify the fugitive emission.

### CROSS-REFERENCE TO RELATED APPLICATIONS

This international PCT patent application claims the benefit of priority to U.S. Provisional Patent Application Ser. No. 63/301,494 (attorney docket MX-2022-PAT-0204-US-PRO), filed on Jan. 21, 2022. The above-referenced patent application is herein incorporated by reference in its entirety.

This application is related to international PCT Patent Application Serial No. PCT/US2020/061407, published May 21, 2021 as WO 2021/102211 A1, which is herein incorporated by reference in its entirety.

### TECHNICAL FIELD

Aspects described herein generally relate to gas detection systems and methods and more specifically to monitoring fugitive gas emissions. Aspects of the disclosure relate to a smart digital platform that collects, analyzes, and renders appropriate information about fugitive gas emissions identified by a sensor network-based emissions monitoring system in a facility.

### DESCRIPTION OF RELATED ART

The concern for clean living, working, and the industrial environment has increased over the recent decades. The United States Environmental Protection Agency (EPA) promulgated, as part of leak detection and repair (LDAR) programs, Method 21 to determine and limit fugitive emissions of gases from industrial facilities (e.g., petroleum refineries, chemical manufacturing facilities, etc.). Fugitive gases may include, but are not limited to, volatile organic compounds (VOCs) and hazardous air pollutants (HAPs). Such LDAR programs are widely adopted in the United States.

The EPA has specified techniques for monitoring/estimating fugitive emissions in a document entitled “Protocol for Equipment Leak Emissions Estimates,” published in November 1995 as EPA-453/R-95-017 (accessible online at https://www3.epa.gov/ttnchiel/efdocs/equiplks.pdf). In general, an industrial facility must conduct manual Method 21-specified inspections at individual components of the facility using a portable gas monitoring equipment (e.g., VOC analyzers) and record the highest measured value for each component. The EPA-specified correlation factors are then applied to the measured values to approximate the total emissions for the facility.

In execution of EPA Method 21, an inspector places an extractive hand-held probe in direct contact with the component under test and traces its circumference, waiting an appropriate amount of time to register a reading of leak concentration (mixing ratio of VOC fraction). If the highest concentration reading is above a control limit, typically 500 to 2000 parts per million, then the component is tagged for repair. The EPA Method 21-determined concentrations are sometimes used to approximate mass flow (or leak) rates through correlation equations to estimate annual emission (leak) rates for the facility-a procedure with several sources of uncertainty. It is well known that manual leak detection methods to monitor and repair sources of fugitive emissions are resource intensive and difficult to apply on hard-to-reach sources. Additionally, EPA Method 21 is expensive to execute and can produce safety concerns for inspectors. This manual inspection procedure only checks a subset of potential emissions points inside a facility and possesses high temporal latency since some components may not be visited for more than a year, creating the potential for a leak to go undetected for an extended time.

Many LDAR programs rely heavily on the EPA's Method 21. As described above, Method 21, however, has a number of drawbacks including: (a) heavy reliance on manual inspections with a portable instrument: (b) extreme inefficiencies (e.g., only a small percentage of all components inspected may have active leaks): (c) safety issues related to manual measurements (e.g., technicians may have to climb towers, may be exposed to inhospitable conditions such as high temperatures, and/or may need to access difficult to reach components): (d) high labor costs; and (e) long time periods between LDAR cycles (e.g., during which large leaks and emissions may remain undetected). For example, due to the infrequent monitoring schedule, some large leaks may not be detected in a timely manner and, therefore, the total emissions estimations may not be accurate.

In view of the foregoing, various solutions (e.g., systems, platforms, and methodologies) have been developed which seek to overcome some or all the aforementioned drawbacks. One such solution is a smart digital platform that collects, analyzes, and/or renders appropriate information about fugitive emissions identified by a sensor network-based emissions monitoring system, also known as a leak detection sensor network or “LDSN”, in a facility. Information regarding such a platform and LDSN can be found in International Patent Publication No. WO/2020/237112, published on Nov. 26, 2020, and International Patent Application No. PCT/IB2021/056932, filed on Jul. 29, 2021, which are incorporated herein by reference. A general description of this solution follows.

FIG. 1 provides an illustration of a wireless sensor network at a plant or process unit. Sensors, depicted as stars in FIG. 1, are fixed in place in the plant or process unit in an optimized way that provides full (or at least substantially full), three-dimensional detection coverage of LDAR (“Leak Detection and Repair”) components within the unit. While some sensors are described as being fixed in place, they can obviously be moved with effort if desired, but are intended to stay in their fixed place over time, and thus may be referred to throughout this document as fixed sensors.

For a given size of a chemical facility or process unit, a minimum number of fixed sensors must be installed to provide full coverage of components under the LDAR monitoring requirement. Due to the high costs of equipment and installation, the density of fixed sensors should be limited to only what is necessary. For example, fixed sensors are spaced about 80-200 feet apart. As a result, average potential source locations (PSLs) created by the sensor system are typically relatively large, e.g., about the size of individual sensor coverage. Finding leaks and particularly small leaks within a PSL has thus proven to be extremely challenging and tends to incur substantial man-hours because there are usually hundreds to thousands of components within each PSL. At present, a technician is dispatched to the field to scan the whole area for possible leaks with a portable gas sniffer, and when one leak or more leaks are found, the technician stops further leak investigation. In many cases, leaks that are found are not the ones that have caused the leak detection. Finding leaks and particularly small leaks is extremely challenging, not to mention finding the leaks that actually triggered the detection notification. As a result of the foregoing, further improvements and solutions are desired toward being able to identify/pinpoint a leak more quickly.

### BRIEF DESCRIPTION OF THE DRAWINGS

The present disclosure is illustrated by way of example and not limited in the accompanying figures in which like reference numerals indicate similar elements and in which:

FIG. 1 is a prior art illustration of a representative facility with a sensor network, in accordance with various aspects of the disclosure;

FIG. 2 illustrates an output of a sensor over a period of time, in accordance with various aspects of the disclosure;

FIG. 3 illustrates how a PSL is created by analyzing data from the sensor network including gas and wind data, in accordance with various aspects of the disclosure;

FIG. 4A shows an example graphical user interface (GUI) of the PSL as it is updated/refined and then displayed on a user computing device, in accordance with various aspects of the disclosure; and FIG. 4B illustrates a mobile sensor with wireless communication circuitry in accordance with various aspects of the disclosure: FIG. 4A and FIG. 4B are collectively referenced as FIG. 4:

FIG. 5 is a flowchart which illustrates the workflow/methodology for a leak source location investigation, in accordance with various aspects of the disclosure;

FIG. 6A and FIG. 6B are illustrations of a first variation of the placement of “mobile” sensors within a PSL, using five (5) “mobile” sensors, in accordance with various aspects of the disclosure; and FIG. 6C and FIG. 6D illustrate measurements read by each of five illustrative sensors over a period of time, in accordance with various aspects of the disclosure: FIG. 6A, FIG. 6B, FIG. 6C, and FIG. 6D are collectively referenced as FIG. 6;

FIG. 7A and FIG. 7B are illustrations of the first variation of the placement of “mobile” sensors within a PSL, using nine (9) “mobile” sensors, in accordance with various aspects of the disclosure: FIG. 7A and FIG. 7B are collectively referenced as FIG. 7; and

FIG. 8 is illustrations of a second variation of the placement of “mobile” sensors within a PSL, in accordance with various aspects of the disclosure.

FIG. 9 illustrates a system for refocusing a technician equipped with a network of mobile sensors and other apparatuses at an industrial facility, in accordance with various aspects of the disclosure.

### DETAILED DESCRIPTION

While the disclosure may be susceptible to embodiment in different forms, there is shown in the drawings, and herein will be described in detail, specific embodiments with the understanding that the present disclosure is to be considered an exemplification of the principles of the disclosure and is not intended to limit the disclosure to that as illustrated and described herein. Therefore, unless otherwise noted, features disclosed herein may be combined to form additional combinations that were not otherwise shown for purposes of brevity. It will be further appreciated that in some embodiments, one or more elements illustrated by way of example in a drawing(s) may be eliminated and/or substituted with alternative elements within the scope of the disclosure.

Methods and systems are disclosed for inspection of a potential source location (PSL) at an industrial facility that has physical components, such as pipes and valves, that transport one or more gaseous materials. A computing device assist in refocusing a technician equipped with a network of mobile sensors and a handheld device (e.g., handheld wand) to detect a gaseous emission at the industrial facility. The mobile sensors capture measurements on a recurring basis to assist the technician responsible for operating the handheld device to identify exactly which physical component located within a PSL at the industrial facility are causing fugitive emissions. The disclosed system and method update an initial PSL by considering the recurring measurements and/or other inputs (e.g., weather data) to reduce the area of the initial PSL, thus better instructing the mechanic to identify the fugitive emission.

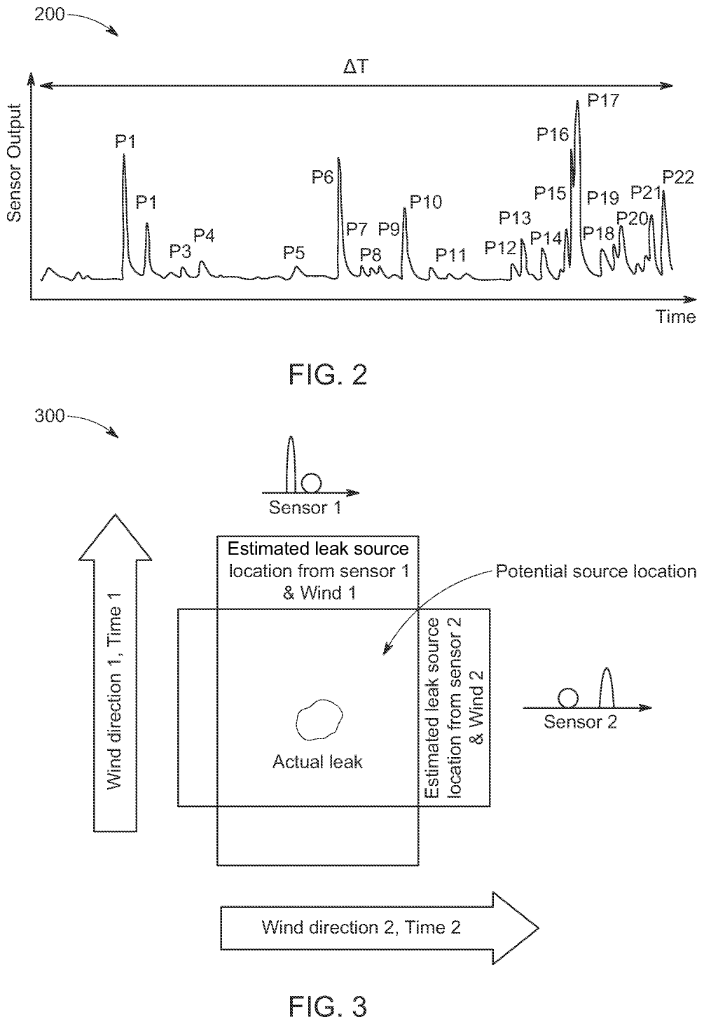

FIG. 2 provides the output of an individual sensor over a period of time. Each peak is a representation of a plume detection. As noted, the graph identifies a number of peaks (P1-P22) over the period of time displayed on the graph. Sensor detect strength, measured as a peak area, is indicative of the plume size at the sensor location.

The raw spatial and temporal gas and wind data with a time stamp from each of the sensor nodes in the LDSN is continually transmitted to the cloud 24 hours a day, 7 days a week and the data is continuously processed in the background using a data analytic algorithm, which was developed to identify the occurrence of fugitive emissions within a facility and estimate the most probable locations of the emission sources. Just as with gas chromatography (GC), the algorithm used by the LDSN first performs baseline modeling/curve-fit to the time-resolved gas sensor output data, and then identifies excursions above the modeled baseline as detection peaks by using a threshold of signal-to-noise ratio S/N≥3. In other words, the baseline itself is not tied to leak detections. Only detection peaks >3 times the noise level are considered detection events. Signal characteristics including the amplitude, width and centroid of each detection peak are then calculated and recorded.

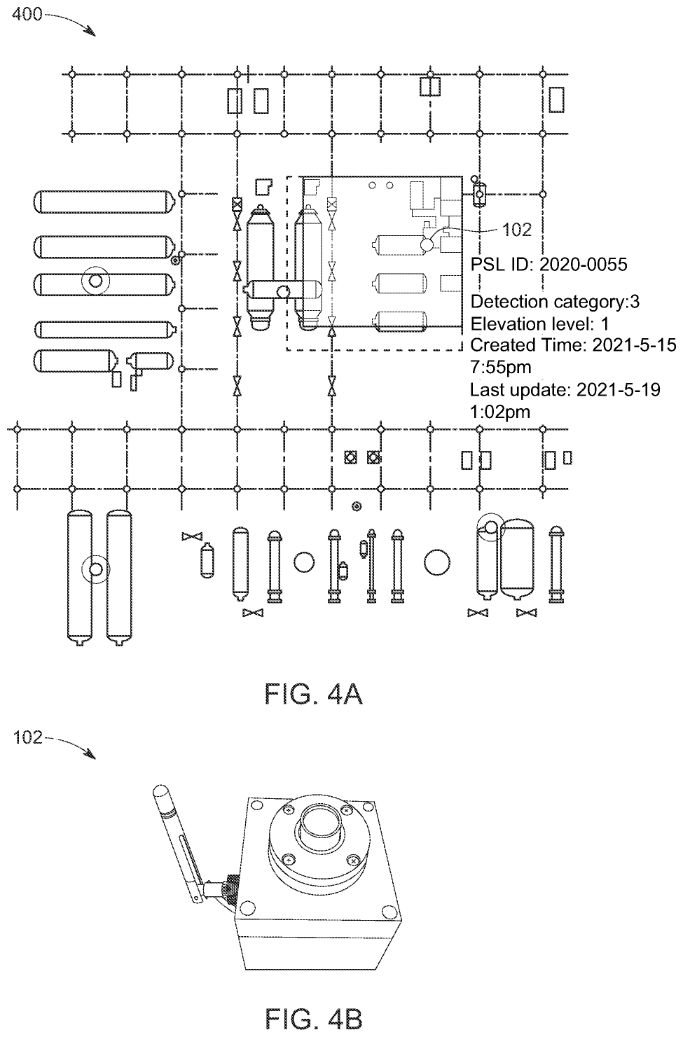

Concurrent with gas sensor signal processing, the algorithm looks for wind direction at the time of each detection. If a detection occurs with a south wind for example, the algorithm assumes a possible leak source to be located to the south of the sensor. When multiple sensors in one vicinity detect emissions under changing wind directions, there will be overlapped areas in the algorithm's leak location estimation. As illustrated in FIG. 3, Sensor 1 shows a detection peak when a south wind is present, thereby the algorithm estimates a potential leak area south of Sensor 1. Next, Sensor 2 shows a detection peak under west wind, but Sensor 1 shows no detection, so the algorithm estimates a potential leak area on the west side of Sensor 2. An area/volume that overlaps corresponds to a potential source location (“PSL”) of the leak.

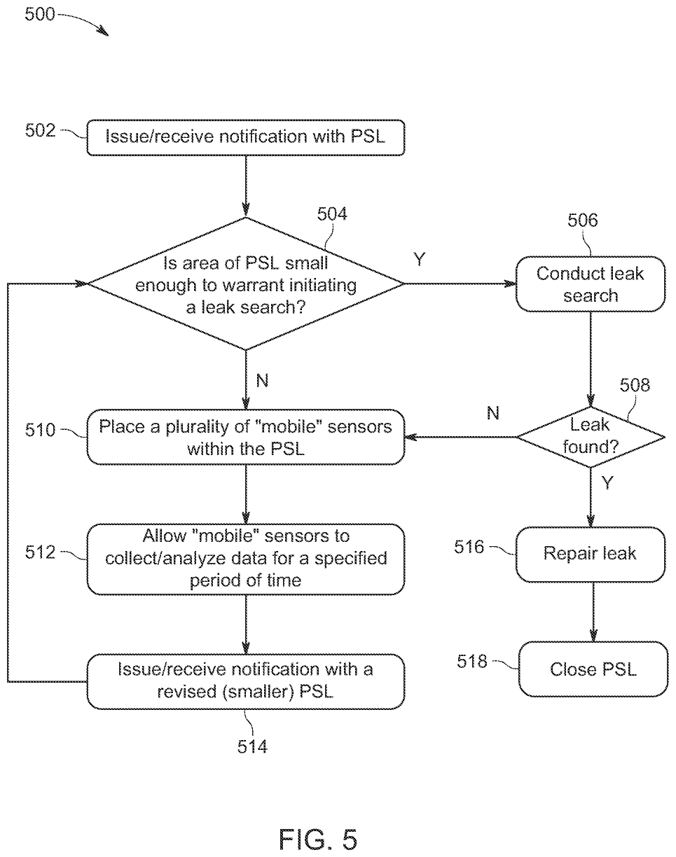

The algorithm continually estimates PSLs from the collaboration of sensors in the LDSN under varying wind conditions and superimposes all the nearby estimated areas/volumes to obtain the most probable PSL. When the number of leak location estimation overlaps hit a preset threshold value within a given time window, e.g., 100 times over a rolling 3-hour window, a notification is issued with the most probable PSL and the detection level. As a part of the notification, the PSL may be represented in any manner including, for instance, in the form of a two- or three-dimensional box. FIG. 4A provides an illustrative example of a PSL represented in the manner of a two-dimensional box.

Once a notification with a PSL and a detection level has been issued, a leak investigation may be scheduled within a predetermined period of time after receipt of the notification (e.g., within 2-15 days or any other time period). At the scheduled time, a technician will be deployed to the PSL in an attempt to pinpoint exactly where the leak is located within the unit. Once the leak source is identified, the leak can be repaired and the PSL can then be closed.

In addition, FIG. 4A illustrates an example graphical user interface (GUI) 400 of the PSL as it is updated/refined and then displayed on a user computing device where the larger box (in dashed line) constitutes the original PSL, and the smaller box (in shading inside the dashed-line box) constitutes the updated/revised PSL. A technician/operator deployed to the PSL at the facility to search for the leak source may be provided, in some examples, with a visual, color-coded map of the updated PSL. In one example, a heat map over the area of the PSL at the facility may be visually displayed on an electronic display of a handheld, user computing device which is provided to the operator/technician.

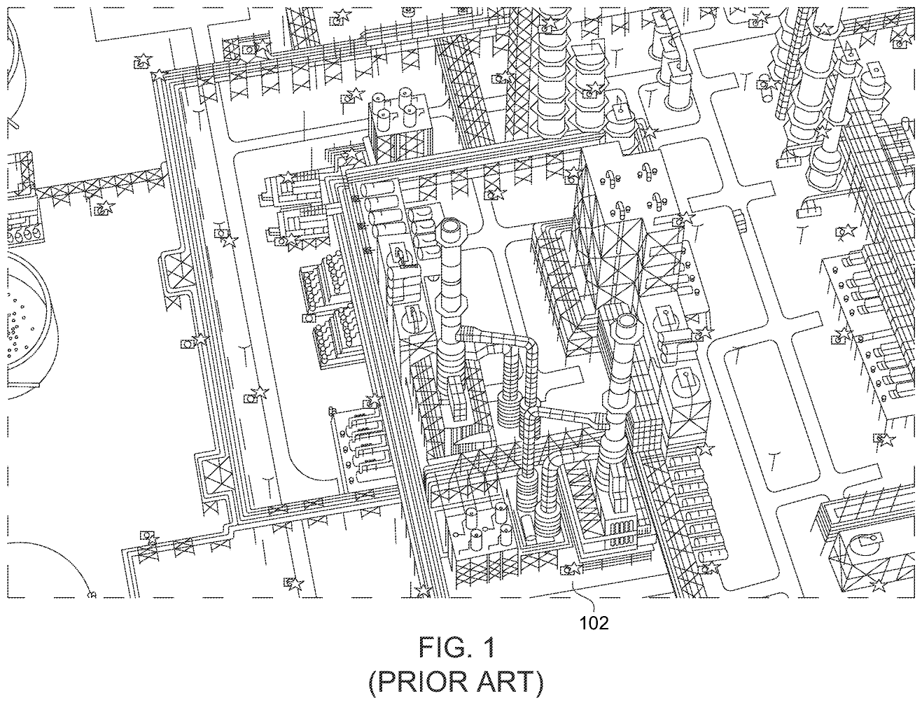

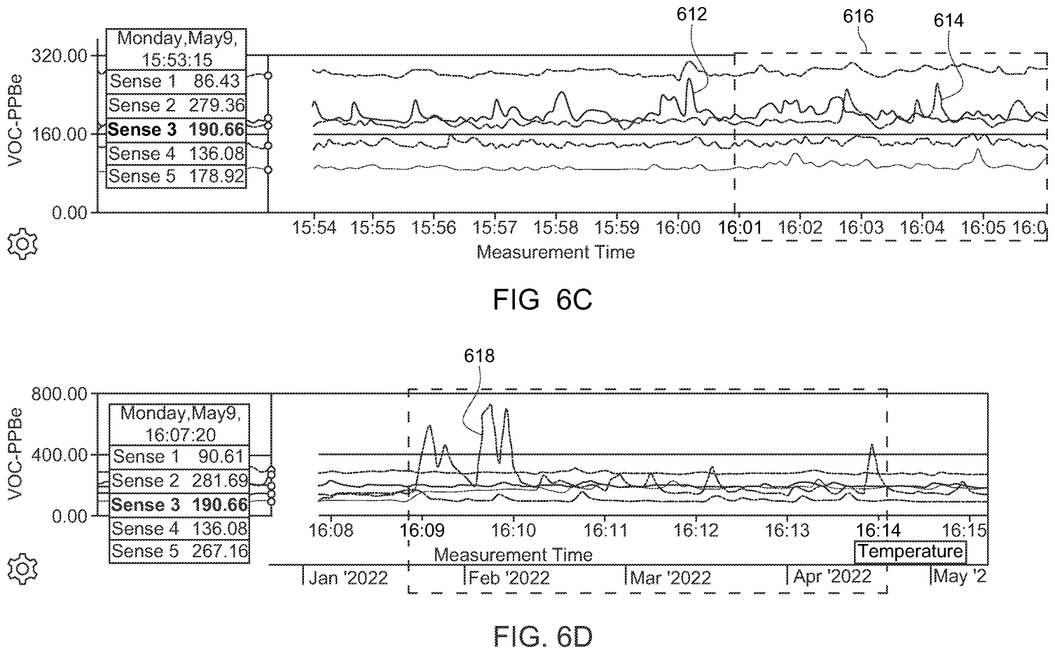

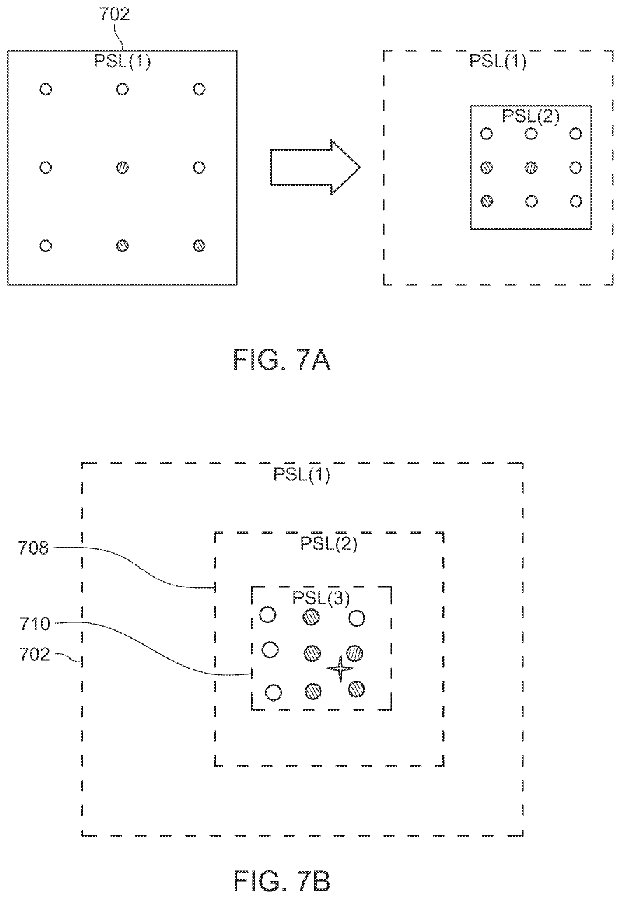

Gas sensors 102 in the sensor network may be placed in the facility in an optimized way that provides full (or at least substantially full) three-dimensional (3D) detection coverage of LDAR components within a facility, as shown in FIG. 1. The 3D considerations at the facility may include accommodating the vertical height relative to ground level (both above and below) so that an updated PSL might be transformed into a smaller cubic footage than an initial PSL. With sensors 102 depicted as stars in FIG. 1, a higher sensor density may provide better leak detection results or even allow construction/representation in a 3D space. The density of sensors is determined by the coverage area of individual sensors, which is determined by the sensitivity of the sensors to the gas streams being monitored. For example, in a crude oil refining unit, each sensor may have a 50-75 foot coverage radius. Readings of gas from one or more sensors indicate gas leaks in that area. However, from a practical perspective, reasonable sensor densities still leave a large, cubic square footage for a technician to manually search for a leak source. In some examples, a facility may be equipped with an array of sensors, but even then, the facility might only have sensors each spaced no less than ten feet (or thirty feet, or other set distance) apart. In other examples, the sensor density may be even farther spaced out, although the density need not be uniform and/or evenly spaced. In any event, the practical spacing of the sensors means that an operator physically investigating an area of a facility for fugitive emission sources would benefit from a smaller PSL area. A network of sensors, as illustrated in FIG. 6 and FIG. 7, may be used to detect gas leaks to help triangulate sensor detections to the source of leaks.

A detection zone of a sensor may be depicted in various ways. In some examples, the detection zone may be altered to accommodate the one or more structures, obstructions, and/or openings in the facility. For example, in a 3-dimensional digital representation, the height of an obstructing structure may have direct bearing on sensor placement, specifically whether the height of a structure is such that a sensor placed at a location may be futile to detect a gaseous plume originating from the opposite side of the obstructing structure. Moreover, the detection zone of a sensor may be affected by the type of sensor being used, the sensitivity of the sensor to a particular gas compound, etc.

Referring to FIG. 4B, a wide variety of sensor technologies may be used for gas detection in various examples described herein. The mobile sensor 102 comprises wireless communications circuitry including an illustrated antenna to transmit measurement data and/or other data to a computing device. The mobile sensor 102 also includes a battery compartment configured to store electrical charge (e.g., a Li-ion battery, a detachable rechargeable battery, an undetachable built-in battery installed in the battery compartment). The mobile sensor 102 may, in some examples, comprise a location tracking module to assist in triangulating the specific location of the mobile sensor.

Gas sensors may comprise electrochemical sensors, infrared sensors, catalytic bead sensors, metal oxide semiconductor (MOS) sensors, photoionization detectors (PIDs), flame ionization detectors (FIDs), thermal conductivity sensors, colorimetric sensors, sensors based on passive sampling techniques, and/or any other sensors configured to measure concentrations of VOCs and/or other hazardous gases.

Detecting gases in open air requires high sensitivity, typically at concentrations in parts per billion (ppb) level, and fast response times (e.g., due to possible wind and changes in wind speed and direction). Several sensor technologies such as MOS and PID meet the requirements and may be used in the emissions monitoring application.

A PID is equipped with a high energy ultraviolet (UV) lamp and electrodes. Gas molecules with low ionization energy entering a UV chamber in a PID are ionized. Resultant ions flow toward a collecting electrode giving rise to an electric current that is directly proportional to the concentration of the gas. Depending on the target gas to be measured, a PID may use a 9.6 eV, 10.0 eV, 10.2 eV, 10.6 eV, or 11.7 eV lamp. The higher the lamp energy, the more gas species can be measured. A lower energy lamp may be preferred for measurement of aromatic compounds (e.g., benzene) because of better specificity.

Gas sensors have varying sensitivities to different gas species and sometimes need to be calibrated properly before use. A surrogate gas of known concentration may be used to calibrate the sensor. For measuring other gases, a cross-sensitivity factor called response factor may be used to correct a sensor output to provide a measurement. For example, isobutylene is typically used for calibrating PIDs due to its moderate sensitivity and low toxicity. When measuring isobutylene concentration, the calibrated PIDs may directly provide a measurement of the concentration. For other gases, a response factor may be used by an emissions monitoring platform for determining the concentration based on measurements provided by the isobutylene-calibrated PIDs.

In addition, in some examples, wind sensors may also be used in the sensor network to help triangulate sensor detections to the source of leaks. In lieu of or in addition to wind sensors, an input feed from an external source about meteorological conditions at the industrial facility may provide the weather information used to transform the initial PSL to the updated PSL.

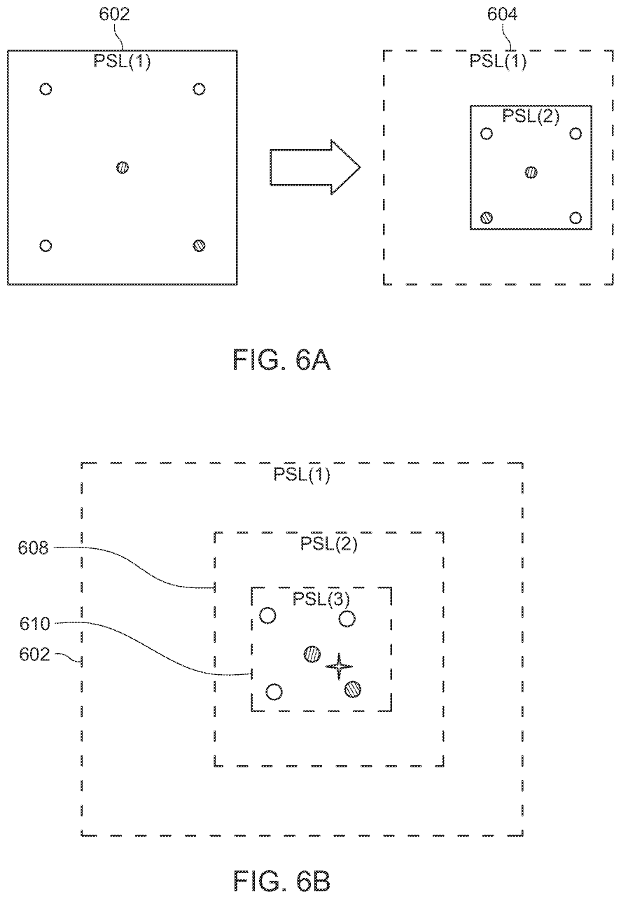

An improved and novel workflow/methodology of conducting a leak source location investigation is described with reference to FIG. 5-FIG. 8. FIG. 5 is a flowchart which illustrates the workflow/methodology as will be described herein.

Referring to FIG. 5, the workflow/methodology 500 begins with a notification 502 with a PSL having been issued/received (see, e.g., FIG. 4A). Once the notification with PSL has been issued/received, a determination 504 is made as to whether the area of the PSL is small enough to warrant initiating a leak search. While it is certainly possible that the PSL will be small enough to warrant initiating a leak search, it is more likely that the probability of identifying the leak location within the PSL issued/received is extremely low, whether it be because the PSL encompasses too large of an area and/or because the PSL encompasses too many components to be checked.

In the event where the PSL is small enough to warrant initiating a leak search, a leak search 506 can be conducted and, if a leak is found 508, the leak can be repaired 516 and the PSL can be closed 518. However, if the leak cannot be found, then the workflow/methodology can follow the workflow/methodology where the PSL is not small enough to warrant initiating a leak search, as described below.

Referring to FIG. 5, in the event where the PSL is not small enough to warrant initiating a leak search, the workflow/methodology has a plurality of “mobile” sensors placed 510 within the PSL. These “mobile” sensors are intended to be differentiated from the “fixed” sensors because, unlike the “fixed” sensors, these “mobile” sensors are intended to be placed in different locations within a single PSL (as will be described herein) and/or to be placed in different PSLs at different times. The “mobile” sensors used in this application must be able to transmit data wirelessly (whereas the “fixed” sensors may be able to transmit data either wirelessly or along wired connections). There are multiple ways of sending data wirelessly, such as Wi-Fi, Bluetooth, cellular, and radio (e.g., 900 MHz). Many chemical plants have Wi-Fi coverage and, therefore, it is relatively easy to onboard sensors through Wi-Fi. Transmission of data through a cellular gateway or cellular tower is also a good option because it bypasses the plant Wi-Fi which often has security firewalls. For example, the wireless communication circuitry (e.g., antenna and modem) in the sensor may directly transmit measurements to one or more data stores without infiltrating the plant's security firewall. Bluetooth connections could also be used so long as the typical Bluetooth range of approximately 30 feet (10 meters) is acceptable, but it is to be understood that the maximum communication range will vary depending on obstacles (person, metal, wall, etc.) or electromagnetic environment. These “mobile” systems may also operate through the same sensor network as the “fixed” sensors or may operate through a separated sensor network.

Once the plurality of “mobile” sensors are placed within the PSL, the “mobile” sensors can analyze 512 pertinent gas data for a specified period of time and, at the completion of the specified period of time, a revised (smaller) PSL can be issued/received 514 and again a determination as to whether the area of the PSL (the revised (smaller) PSL) is small enough to warrant initiating a leak search, and the workflow/methodology with reference to FIG. 5 can be repeated.

Numerous variations within the general workflow/methodology as described with reference to FIG. 5 are provided. A first variation relating to the placement of the plurality of “mobile” sensors within the PSL is provided with reference to FIG. 6 and FIG. 7.

FIG. 6A and FIG. 7A illustrate placing a plurality of “mobile” sensors evenly within the original PSL (illustrated as PSL (1) in 602), where FIG. 6A illustrates placing five (5) “mobile” sensors and FIG. 7A illustrates placing nine (9) “mobile” sensors (as shown by PSL (1) 702). Of course, it is to be understood that any amount of a plurality of “mobile” sensors (e.g., two (2), five (5), nine (9), twelve (12), etc.) may be placed within the original PSL, with the actual number to be placed within the original PSL varying on a number of factors (e.g., the size of the PSL, the number of “mobile” sensors available, actual sizes or shapes of components and structural impedance and/or available platform for sensor placement within the original PSL, etc.). Placing the plurality of “mobile” sensors within the original PSL will likely result in one or more of the “mobile” sensors detecting higher concentrations of gas than the others, such that a revised (smaller) PSL will generally be provided around those “mobile” sensors (e.g., in a “hot spot”).

Once the plurality of “mobile” sensors results in a revised (smaller) PSL being issued/received, and if the area of the revised (smaller) PSL is not small enough to warrant initiating a leak search, the plurality of “mobile” sensors can be moved (as shown in the updated positioning 604) and placed evenly within the revised (smaller) PSL (illustrated in FIG. 6B as PSL (2) 608 and FIG. 7B as PSL (2) 708) until a further revised (smaller) PSL is issued/received (illustrated in FIG. 6B as PSL (3) 610 and FIG. 7B as PSL (3) 710). This workflow/methodology can continue until the PSL is sufficiently small to locate the actual leak source (illustrated by a star symbol in each of FIG. 6B and FIG. 7B).

Referring to FIG. 6C and FIG. 6D, these illustrate measurements read by each of five illustrative sensors over a period of time. The five illustrative sensors correspond to those illustrated as dots in FIG. 6A and FIG. 6B. In one example, the area of PSL (1) 602 may be sixty feet by sixty feet, and in the PSL 602, sensor Sense1 is represented by the dot in the upper-left corner, sensor Sense2 is represented by the dot in the upper-right corner, sensor Sense3 is represented by the filled-in dot near the center, sensor Sense4 is represented by the dot in the lower-left corner, and sensor Sense5 is represented by the dot in the lower-right corner. FIG. 6C shows the respective measurements collected by each of the five, aforementioned sensors over a period of time, such as five minutes (or other longer or shorter predetermined period of time, such as thirty seconds, sixty seconds, or other duration of time). Each sensor may wirelessly transmit recurring measurement data to one or more data stores that collect/store the data. In particular, a computing device may analyze the recurring measurements over the time period and identify that sensor Sense3 has the highest detection 612 (e.g., peak value) observed/measured over a time interval. As a result, the computing device may cause one or more of the sensors to be physically moved such that the initial PSL 602 is transformed into an updated PSL (2) 604, 608 that occupies a smaller area. For example, if Sense1, Sense2, Sense4, and Sense5 are move halfway towards Sense3 near the center of the initial PSL 602, then the updated PSL (2) 608 is ¼ of the size of the initial PSL (1) 602.

In one example, when the computing device determines that the updated PSL 608 still exceeds a maximum area threshold, then the computing device may hold on instructing a technician to operate a handheld gas measurement/detection device (e.g., a handheld wand) on or near physical components. Even though the overall area to be manually scanned by the technician has been reduced to that region that overlaps with the updated PSL 608 and the initial PSL 602, but to the exclusion of the area that is outside of updated PSL 608, iterating through the steps of the method may further narrow the area to be searched. Next, recurring measurements are collected for an additional period of time 616 (e.g., five minutes or other longer or shorter predetermined period of time, such as thirty seconds, sixty seconds, or other duration of time) to identify the sensor that has the highest peak measurement. For example, in FIG. 6C, during time period 616, sensor Sense5 measures the highest detection 614 (e.g., peak value). Moreover, sensor Sense3 612 also observes a high level of detection. As a result, as shown in FIG. 6B, the sensors are positioned again so that sensors for Sense1, Sense2, Sense3, and Sense4 are moved halfway towards the sensor for Sense5 with the center between the previous positions of the sensors for Sense3 and Sense5. The new PSL 610 is ¼ of the area of the previous PSL 608 or 1/16 of the initial PSL 602. At this stage in the process of updating the PSL, the area consumed by the PSL 610 is small enough to satisfy the maximum area threshold. Therefore, the computing device may provide the final PSL 610 to a user computing device (e.g., a smartphone or mobile tablet) of the technician to refocus the technician to operate a handheld device (e.g., handheld wand) on the physical components located within the PSL 610. In some examples, the user computing device may further convey which sensor (e.g., sensor corresponding to Sense4 618) measured the highest level of detection so the technician can start the manual searching process in its vicinity first. The maximum area threshold may be subjectively set by an industrial facility, a technician, or by industry-accepted standards, and need not necessarily be the same value in all implementations.

Regarding FIG. 7, similar to FIG. 6, any number of sensors may be arranged in an area of an industrial facility as illustrated in PSL (1) 702. While FIG. 7 shows nine sensors arranged in a evenly-spaced arrangement in the area, any number of sensors (e.g., 3 or more sensors) may be placed in the area to triangulate a more precise location of an emission leak from a physical component. Moreover, while some examples reference a 2-dimensional depiction of a PSL, the disclosure is not so limited. Rather, as illustrated in FIG. 1, the PSL may be defined in a 3-dimensional environment where the refining and/or narrowing of PSL is in square footage or even cubic footage that takes into account that the PSL may have one or both of horizontal dimensions and/or vertical dimensions that extend above and/or below ground level. In addition, although the PSLs are sometimes graphically depicted as rectangular in shape, the disclosure is not so limited. Rather, the PSL may take any one of several different shapes including but not limited to a circular shape with the centroid of the shape aligned with a sensor 102, or a non-traditional shape with boundaries that are customized by a computing device that calculates a PSL based on numerous inputs. For example, the shape of the PSL boundary need not be a square/rectangle, and it may be round/elliptical or other non-traditional shapes to accommodate for obstructions or other reasons.

In one example, after a notification with PSL is issued/received, the position/placement of sensors in the area of the leak detection sensor network (LDSN) may be reconfigured as illustrated in FIG. 7A to continually update/refine the PSL as the algorithm continues running in the background of a computing device (with a computer processor and a memory). Thus, the PSL will continue to be updated/refined with more data to a smaller, more precise, and more accurate location, as illustrated by PSL (1) 702, PSL (2) 708, and PSL (3) 710 that are purporting to focus an operator/technician on an area that is more likely to contain the physical component emitting fugitive emissions (denoted by the star-like icon in PSL (3) 710). For example, the technician may be instructed to start operating the handheld device/sensor/wand on those physical components located within the updated PSL (e.g., PSL (3) 710) and not on those physical components outside of PSL (3) but within the initial PSLs (e.g., PSL (1) 702 and PSL (2) 708. The instructions to the technician may be generated on an electronic display of a handheld, user computing device which is provided to the operator/technician. The optimized area that the technician can begin searching provides a meaningful efficiency benefit.

While some examples illustrate the PSL as being caused to be rearranged by the computing device to more confidentially include the source of the leak. However, in some examples, the physical component with the leak may be ultimately found outside of an updated PSL. For example, in some examples, the method progressively shrinks the PSL by placing mobile sensors at the corners and center of PSL and then repositioning them around the one or more sensors that are measuring levels of detection over a threshold (e.g., high levels of detection). Nevertheless, the updated PSL provides a technician with a beneficial starting point from which to start manual searching for a leak and progressively search outwards from the sensor with the highest measurements. In one example, the disclosed system contemplates that a gas source may be confirmed in the proximity of a sensor but outside of the update PSL.

Of course, in some examples, regulatory and/or compliance requirements may dictate that an initial leak notification that triggers an operator/technician to be deployed to investigate an initial PSL cannot be cleared/closed if the PSL is updated to include components located outside of the initial PSL and one of those particular components are found to have a leak that is subsequently repaired. In any event, regulatory and/or compliance requirements aside, the disclosure contemplates that a more precise triangulation of a PSL is possible with the processing of ongoing/recurring mobile sensor measurements over time more varying locations. Moreover, in some embodiments weather/meteorological data may be included to further refine the precise updating of sensor placements. For example, a database may store weather data corresponding to wind speed and wind direction near the PSL at an industrial facility and cause the computing device to instruct movement of sensors from an initial PSL to an updated PSL based on the weather data, including but not limited to wind data (e.g., wind speed and wind direction) and other meteorological properties.

Of course, it is to be understood that the five (5) “mobile” sensors illustrated in FIG. 6A and FIG. 6B and the nine (9) “mobile” sensors illustrated in FIG. 7A and FIG. 7B are just exemplary. Further, it is to be understood that the number of “mobile” sensors placed in PSL (1) does not need to equal the number of “mobile” sensors placed in PSL (2) and/or PSL (3).

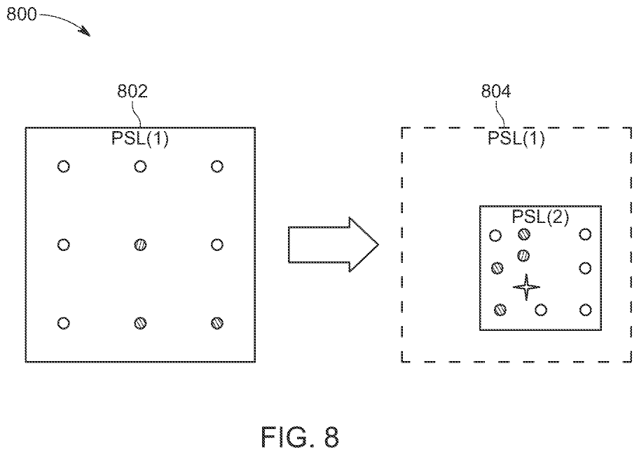

A second variation relating to the placement of the plurality of “mobile” sensors within the PSL is provided with reference to FIG. 8. FIG. 8 illustrates placing a plurality of “mobile” sensors evenly within the original PSL (illustrated as PSL (1)), wherein FIG. 8 illustrates placing nine (9) “mobile” sensors). Once a revised (smaller) PSL is generated or the “mobile” sensor(s) that shows the highest detection level is determined, those “mobile” sensor(s) can be left in their original positions and the other “mobile” sensors are relocated (preferably as evenly as possible) around the “mobile” sensors left in their original position, until a further revised (smaller) PSL is issued/received. This workflow/methodology of leaving one or more sensors in their locations and relocating the other sensors can continue until the PSL is sufficiently small to locate the actual leak source (illustrated by a star symbol in FIG. 8).

For the workflow/methodology as described with reference to FIG. 5, the plurality of “mobile” sensors must be equipped with a high sensitivity gas sensor. The plurality of “mobile” sensors preferably have a built-in anemometer and GPS tracking. In an embodiment, the plurality of “mobile” sensors may be onboarded into the LDSN such that the sensors automatically send their locations once placed. Alternatively, in another embodiment, the identification and location of the plurality of “mobile” sensors may be manually entered into the LDSN through a mobile app (or the like) in the field. In another embodiment, the plurality of “mobile” systems may operate through a system other than the LDSN.

The “mobile” sensors and/or the LDSN and/or any other system utilized, also preferably collects/analyzes wind data in conjunction with the gas data in order to determine the probable location of the leak (as discussed above with reference to FIG. 3). The wind data can come from the meteorological data used for the LDSN in the particular unit, or from a mobile wind sensor (which could be associated with the “mobile” sensors discussed herein or could be separate stand-alone mobile wind sensors). Mobile wind sensors in a sensor network will typically provide more accurate local wind data as they would provide wind data directly from the PSL.

As shown in FIG. 6, FIG. 7, and FIG. 8, the “mobile” sensors are shown as circles and the detection level for each can also be depicted, e.g., the darker the circle, the higher the detections. However, it is to be understood that any appropriate/desired indicators for the “mobile” sensors can be utilized and that any appropriate/desired indicators for the level of detections can be utilized.

Further, while the placement of the “mobile” sensors is sometimes evenly distributed as discussed, it is to be understood that it may not be feasible/possible to evenly distribute the “mobile” sensors within a PSL due to various issues. For example, some perceived sensor locations have no accessible platform and they would require scaffolding. Some components such as fan banks and heat exchanges are very large in size and they may happen to be located in an “optimal” sensor location. Some areas such as the top of a fuel vessel are classified as restricted area, and such a “mobile” sensor could not be placed there. Furthermore, it is to be understood that while even distribution of the “mobile” sensors within a PSL is preferred, it is not an absolute requirement as the “mobile” sensors could be randomly placed within the PSL or, depending on the PSL, the “mobile” sensors could be stacked more in one area than another if it is known that certain areas of the PSL do not contain components that are likely to leak. For example, FIG. 8 illustrates that an initial PSL 802 of evenly spaced arrangement of sensors may be updated into a PSL 804 of unevenly spaced sensor arrangement based on the transformation calculated by the computing device.

Further, in some instances, it may be possible to include a single “mobile” sensor within the PSL and have the single “mobile” sensor collaborate with the “fixed” sensors in the facility in order to further refine the PSL with the assistance of the single “mobile” sensor. Also, in such a scenario, multiple “mobile” sensors could collaborate with the “fixed” sensors in the facility in order to further refine with PSL.

Thus, the workflow/methodology as illustrated in FIG. 5 provides an improved and novel workflow/methodology for leak source location investigations, as it allows for the substantial reduction in the size of the PSL which must be searched in order to identify/pinpoint the source of the leak more quickly, efficiently, and economically, especially when the original PSL may have a large area and/or contains a large number of components.

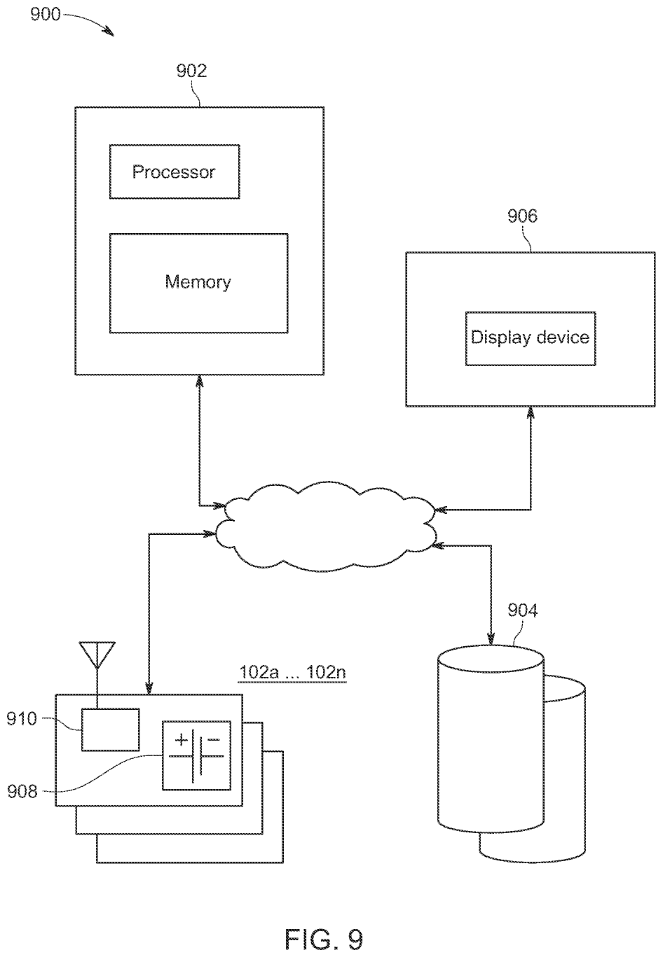

Regarding FIG. 9, a system 900 is disclosed for refocusing a technician equipped with a network of mobile sensors and a handheld device (e.g., handheld wand/sensor) to detect a gaseous emission at an industrial facility that has physical components that transport one or more gaseous materials. A computing device 902 comprising at least one computer processor and memory is communicatively coupled through a database interface (e.g., over a network as depicted by the cloud in FIG. 9) to one or more data stores 904. The data stores 904 store, inter alia, recurring measurements received from one or more (e.g., at least three) sensors 102 positioned at the industrial facility. The sensors are mobile sensors that can be readily moved from one position to another position based on instructions generated by the computing device 902 and sent to a user computing device 906 with a display screen/device operated by the technician. The plurality of sensors 102, 102a . . . 102n each include a battery compartment 908 configured to store electrical charge, and a wireless communication circuitry 910 configured to transmit measurements. The battery compartment 908 may be just a casing with a positive pole and negative pole to connect to a removable battery, or alternatively may be a built-in battery encased inside of the sensor 102. The wireless communication circuitry 910 may communicate using one or more protocols and techniques explained herein with respect to at least FIG. 5.

The memory in the computing device 902 may store computer-executable instructions that, when executed by the at least one computer processor, cause the computing device to perform one or more steps to refocus a technician at an industrial facility using a network of mobile sensors. At a first time t1, the computing device 902 may provide an initial potential source location (PSL) in a notification that guides the technician. When the mobile sensors 102a . . . 102n are arranged at the locations indicated in the notification of the PSL 602, the mobile sensors collect and store the type of recurring measurement, explained with respect to FIG. 1, FIG. 2, FIG. 3, and FIG. 4. The computing device 902 receives, through an interface, the recurring measurements obtained between a first time t1 and a later time t2. In some examples the time interval from time t1 to time t2 has a duration of at least five minutes, but in other examples, the duration may be longer or shorter, such as thirty seconds, sixty seconds, or other duration of time.

Based on the recurring measurements, the computing device 902 transforms the initial PSL into an updated PSL that occupies a smaller area (e.g., square footage or cubic footage) than the initial PSL. The updated PSL is sent in a notification to the user computing device 906 to cause the sensors 102a . . . 102n to be physically moved to positions corresponding to the updated PSL. The physically moving of the sensors may be done in an automated manner (e.g., when the sensors are equipped with wheels, motors, drone-like capabilities, or other mobility mechanisms) else the technician may manually move them about the industrial facility to the positions indicated in the notification of the updated PSL. In some examples less than all of the sensors may be moved. In other examples, sensors may be moved between 40% to 60% towards the other sensors and not necessarily halfway. Moreover, in some examples, not all sensors are moved by the same distance and some sensors may be shifted more than others, based on the notification generated for the updated PSL. With the additional analysis provided to the computing device 902 from the sensors in an updated PSL arrangement, the technician may refocus the manual search of physical components to start with the updated PSL region that overlaps with the initial PSL. The computing device 902 may provide further notification to the user computing device 906 in real-time as additional analysis is received. For example, the movement of the sensors and the analysis by the computing device 902 need not be performed in serial, and may be performed asynchronously such that as soon as one sensor is moved and confirmed to be in the updated position, it can begin collecting recurring measurements for a desired window of time. Then, as other sensors are moved and put into position, they may also immediately begin the desired measurements. As such, the window 616 in representative FIG. 6C may be a sliding, fragmented window, in some examples.

Although FIG. 9 depicts the computing device 902, data stores 904, and sensors 102a . . . 102n to be separate devices that communicate over a wireless network, the disclosure is not so limited. Rather, in some embodiments, the data store 904 and computing device 902 may be conflated into one system or device. In other embodiments, one or more of the sensors 102a . . . 102n may be edge devices embedded with the computer-executable instructions and processing power to perform the method steps performed by computing device 902, but on the edge device or edge devices itself. As such, one or more sensor 102 may supplant the computing device 902 and communicate directly with one or more data stores 904. In yet another embodiment, the computing device 902, data stores 904, and sensors 102a . . . 102n may all be coalesced into a single device that operates at the industrial facility to communicate directly with a user computing device 906 to communicate the updated PSL and other notifications. In such an embodiment, the wireless communication may be by way of short-range communication such as Bluetooth or other protocols that allow for direct communication without requiring an external gateway or infiltrating the plant's security firewall.

It is also to be understood that the workflow/methodology may also be used to conduct a leak survey without having an original PSL issued/received. For instance, should the presence of gas in a facility be detected by smell, the workflow/methodology could be utilized to place a plurality of “mobile” sensors near where the smell was detected in an attempt to identify/pinpoint the leak source.

While particular embodiments are illustrated in and described with respect to the drawings, it is envisioned that those skilled in the art may devise various modifications without departing from the spirit and scope of the appended claims. It will therefore be appreciated that the scope of the disclosure and the appended claims is not limited to the specific embodiments illustrated in and discussed with respect to the drawings and that modifications and other embodiments are intended to be included within the scope of the disclosure and appended drawings. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the disclosure and the appended claims.

What is claimed:

1. A system for refocusing a technician equipped with a network of mobile sensors and a handheld device to detect a gaseous emission at an industrial facility that has physical components that transport one or more gaseous materials, the system comprising:
at least three sensors positioned at the industrial facility, wherein each of the at least three sensors comprises: (i) a battery compartment configured to store electrical charge; and (ii) wireless communication circuitry configured to transmit a measurement;
one or more data stores storing recurring measurements obtained by the at least three sensors;
a computing device comprising at least one computer processor, an interface to one or more data stores, and a memory storing computer-executable instructions that, when executed by the at least one computer processor, cause the computing device to:
(a) at a time t1, provide an initial potential source location (PSL) in a notification for the technician, who is responsible for operating the handheld device to detect gaseous emissions from a physical component located within the initial PSL at the industrial facility;
(b) receive through the interface, the recurring measurements obtained between time t1 and a later time t2, by the at least three sensors positioned in a vicinity of the initial PSL, wherein a time interval from the time t1 to the time t2 has a predetermined time duration:
(c) based on the recurring measurements, transform the initial PSL into an updated PSL;
(d) cause, after time t2, one or more of the at least three sensors to be physically moved at the industrial facility to positions corresponding to the updated PSL, wherein the updated PSL occupies a smaller area than the initial PSL; and
(e) provide the updated PSL to a user computing device of the technician to refocus the technician to operate after time t2 the handheld device on or near the physical components located within an overlap region of the updated PSL and the initial PSL.

2. The system of claim 1, wherein the wireless communication circuitry of each of the at least three sensors directly transmits the measurement to the one or more data stores using short-range wireless communication.

3. The system of claim 1, wherein the one or more data stores further store current weather data corresponding to wind speed and wind direction at the industrial facility, and wherein the memory stores computer-executable instructions that, when executed by the at least one computer processor, cause the computing device to:
receive through the interface, the weather data, wherein the transforming further comprises using the weather data to transform the initial PSL into the updated PSL.

4. The system of claim 1, wherein as a result of step (d), less than all of the at least three sensors are physically moved.

5. The system of claim 1, wherein the transforming comprises using a peak value measured during the time interval from the time t1 to the time t2 by a first sensor of the at least three sensors, as the measurement transmitted by the first sensor for storing in the one or more data stores.

6. The system of claim 1, wherein the memory stores computer-executable instructions that, when executed by the at least one computer processor, cause the computing device to:
determine that the updated PSL exceeds a maximum area threshold; and
repeat steps (b) and (c) for a later time interval that starts after the time t2 before performing step (e).

7. A computing device for refocusing a technician equipped with a network of mobile sensors and a handheld device to detect a gaseous emission at an industrial facility, the computing device comprising:
at least one computer processor:
an interface to one or more data stores storing recurring measurements obtained by at least three sensors positioned at the industrial facility that has physical components that transport one or more gaseous materials, wherein each of the at least three sensors comprises:
(i) a battery compartment configured to store electrical charge; and (ii) a wireless communication circuitry configured to transmit a measurement; and
a computer-readable memory storing computer-executable instructions that, when executed by the at least one computer processor, cause the computing device to:
(a) at a time t1, provide an initial potential source location (PSL) in a notification for the technician, who is responsible for operating the handheld device to detect gaseous emissions from a physical component located within the initial PSL at the industrial facility;
(b) receive through the interface, the recurring measurements obtained between time t1 and a later time t2, by the at least three sensors positioned in a vicinity of the initial PSL, wherein a time interval from the time t1 to the time t2 has a time duration of at least 30 seconds:
(c) based on the recurring measurements, transform the initial PSL into an updated PSL;
(d) cause, after time t2, each of the at least three sensors to be physically moved at the industrial facility to positions corresponding to the updated PSL, wherein the updated PSL occupies a smaller area than the initial PSL; and
(e) provide the updated PSL to a user computing device of the technician to refocus the technician to operate after time t2 the handheld device on or near the physical components located within an overlap region of the updated PSL and the initial PSL.

8. The computing device of claim 7, wherein the transforming comprises using a peak value measured during the time interval from the time t1 to the time t2 by a first sensor of the at least three sensors, as the measurement transmitted by the first sensor for storing in the one or more data stores.

9. The computing device of claim 7, wherein the memory stores computer-executable instructions that, when executed by the at least one computer processor, cause the computing device to:
determine that the updated PSL exceeds a maximum area threshold; and
repeat steps (b) and (c) for a later time interval that starts after the time t2.

10. The computing device of claim 7, wherein the memory stores computer-executable instructions that, when executed by the at least one computer processor, cause the computing device to:
determine that the updated PSL exceeds a maximum area threshold;
repeat steps (b) and (c) for a later time interval that starts after the time t2; and
after the repeating of steps (b) and (c), determine that the updated PSL meets the maximum area threshold, then repeat step (e).

11. The computing device of claim 7, wherein the at least three sensors in the vicinity of the initial PSL at the industrial facility are each spaced no less than ten feet apart.

12. The computing device of claim 7, wherein the initial PSL is a superset of the updated PSL.

13. The computing device of claim 7, wherein the updated PSL is a smaller cubic footage than the initial PSL, wherein the PSL at the industrial facility is in three dimensions including a vertical height relative to ground level.

14. The computing device of claim 7, wherein the transforming comprises triangulating based on the recurring measurements.

15. The computing device of claim 7, wherein the one or more data stores further store weather data corresponding to wind speed and wind direction near the initial PSL at the industrial facility, and wherein the memory stores computer-executable instructions that, when executed by the at least one computer processor, cause the computing device to:
receive through the interface, the weather data, wherein the transforming further comprises using the weather data to transform the initial PSL into the updated PSL.

16. The computing device of claim 7, wherein the handheld device is a handheld wand, and wherein a display device is coupled to the user computing device, wherein the memory stores computer-executable instructions that, when executed by the at least one computer processor, cause the computing device to:
output, on the display device coupled to the user computing device, instructions to the technician to start operating the handheld wand on the physical components located within the overlap region.

17. The computing device of claim 7, wherein a display device is coupled to the user computing device, and wherein the memory stores computer-executable instructions that, when executed by the at least one computer processor, cause the computing device to:
output, on the display device coupled to the user computing device, instructions to the technician about updated positions in the industrial facility to physical move each of the at least three sensors, wherein the updated positions correspond to the updated PSL.

18. A method comprising:
(a) at a time t1, sending to a user computing device of a technician, an initial potential source location (PSL) in a notification, wherein the technician is responsible for operating a handheld device to detect gaseous emissions from a physical component located within the initial PSL at an industrial facility that has physical components that transport one or more gaseous materials;
(b) receiving, at a computing device from a data store, recurring measurements obtained between time t1 and a later time t2, by at least three mobile sensors positioned in a vicinity of the initial PSL at the industrial facility and stored in the data store, wherein a time interval from the time t1 to the time t2 has a time duration of at least thirty seconds;
(c) based on the recurring measurements, transforming by the computing device, the initial PSL into an updated PSL;
(d) causing, after time t2, one or more of the at least three mobile sensors to be physically moved at the industrial facility to positions corresponding to the updated PSL, wherein the updated PSL occupies a smaller area than the initial PSL; and
(e) sending to the user computing device by the computing device, the updated PSL to refocus the technician to operate after time t2 the handheld device on or near the physical components located within an overlap region of the updated PSL and the initial PSL.

19. The method of claim 18, wherein data store further stores weather data corresponding to wind speed and wind direction at the industrial facility, and the method comprising:
receiving, at the computing device from the data store, the weather data, wherein the transforming further comprises using the weather data to transform the initial PSL into the updated PSL.

20. The method of claim 18, further comprising:
outputting, on a display device coupled to the user computing device, instructions to the technician to start operating the handheld device on or near the physical components located within the overlap region.
outputting, on the display device coupled to the user computing device, instructions to the technician about updated positions in the industrial facility to physical move each of the at least three sensors, wherein the updated positions correspond to the updated PSL.
